(19) United States
(12) Patent Application Publication
(10) Pub. No.: US 2023/0277355 A1
(43) Pub. Date: Sep. 7, 2023

(54) GARMENT-BASED SPLINT SYSTEM

(71) Applicant: TRITON SYSTEMS, INC., Chelmsford, MA (US)

(72) Inventors: Marmaduke J. Loke, Lowell, MA (US); Marya H. Schnedeker, Cambridge, MA (US); Michael J. Guarino, Wakefield, MA (US); William M. Lindsey, Lowell, MA (US); James F. Saunders, Lowell, MA (US); George Santiago, Marlborough, MA (US); Brian E. Moyer, Nashua, NH (US)

(21) Appl. No.: 18/176,938

(22) Filed: Mar. 1, 2023

Related U.S. Application Data
(60) Provisional application No. 63/315,404, filed on Mar. 1, 2022.

(51) Int. Cl. A61F 5/058; A41D 13/00; A41D 13/12

(52) U.S. Cl. CPC A61F 5/058; A41D 13/0012; A41D 13/1236

(57) ABSTRACT

Disclosed is a splint system for protecting and immobilizing an injured body segment and/or associated joint. The splint system is a garment-based splint system including a garment including an inner fabric layer having a skin-facing surface layer and an interior surface layer, and an outer fabric layer, wherein the inner fabric layer and the outer fabric layer define one or more pockets and one or more channels connecting a filler source to the one or more pockets.

### CROSS REFERENCE TO RELATED APPLICATIONS

This application claims benefit of priority to U.S. Provisional Patent application No. 63/315,404 entitled GARMENT-BASED SPLINT SYSTEM filed on Mar. 1, 2022, which is hereby incorporated in its entirety for any purpose.

### GOVERNMENT SPONSORSHIP

This invention was made with government support under contract nos. W81XWH-21-C-0062 awarded by the US Army Medical Research Acquisition Activity (USAMRAA). The government has certain rights in the invention.

### RELATED APPLICATIONS

This application is related to the U.S. Provisional Patent Application 63/315,424, entitled Orthosis System filed on Mar. 1, 2022, and its non-provisional filing which is being filed contemporaneously herewith. Each of which is hereby incorporated by reference in its entirety.

### BACKGROUND

The 2018 National Defense Strategy identified “Forward force maneuver and posture resilience” as a key capability that must be modernized to “solidify our competitive advantage.”

Currently available solutions are less than desirable. Powered exoskeletons are bulky, heavy, expensive, and require complex adjustment to fit an injured person's body. Formable splints and braces are not able to fully immobilize an injured limb, especially during subsequent movement, which increases the risk of re-injury. Vacuum splints are very heavy and render the casualty non-ambulatory. Immobilizing walking boots are large and heavy. Simple braces and wraps can effectively treat minor sprains but are not well suited for more severe injuries. Air-casts require additional hardware (a pump) to apply and are not designed for use in austere environments. Plaster and or fiberglass casts require advanced skills to apply, copious pack volume, and significantly more time to apply and set than this garment. Pinned fixators require advanced training and specialized tools to apply, at times including imaging equipment, and necessitate puncturing the skin which can lead to additional complications.

Thus, new and improved injury support systems are needed that:

Improve warfighter resilience and survivability, in conjunction with orthotic elements, allowing injured warfighters to return to mission safely and effectively despite having sustained lower-limb injuries that would otherwise diminish their capabilities by reducing undesirable motion of an injured limb near the ankle.

Improve medic effectiveness by reducing pack footprint, required application time, training, and needed skill compared to the current standard of care.

Improve unit effectiveness by promoting buddy-care (or possibly even self-care) to reduce task burdens on medics allowing them to respond to injuries requiring expert care.

### SUMMARY

The disclosure describes a garment-based splint system. Some embodiments comprise a garment comprising an inner fabric layer having a skin-facing layer, an interior fabric layer, and an outer fabric layer, wherein the inner fabric layer and the outer fabric layer define one or more pockets and one or more channels connecting a filler source to one or more pockets.

Some embodiments further comprise a one way quick disconnect at a distal end of at least one of the one or more channels adapted for connection with a filler source to allow filler to flow through the one or more channel into one or more pocket, and to allow removal of the filler source.

In some embodiments, the garment is shaped and configured to stabilize an upper limb or a lower limb or other body component such as the neck.

Some embodiments provide a kit for a garment-based splint system, the kit comprising: a garment comprising an inner fabric layer having a skin-facing surface and an interior surface, and an outer fabric layer, wherein the inner fabric layer and the outer fabric layer define one or more pockets and one or more channels connecting a filler source to the one or more pockets; and a filler source.

In some embodiments, this garment may be supplemented with more rigid components for additional support and movement restriction.

In some embodiments, this garment may include pass throughs regions for access to underlying critical tissue regions (e.g., tracheal access).

In some embodiments, this garment may have pockets that are separate from the skin barrier layer. These pockets may be attached and aligned with the garment and underlying musculoskeletal system via reusable attachment techniques such as hook and loop fasteners or elastic wrapping materials/straps.

In some embodiments the filler material (e.g., foam) is integrated with additional components within or separate from the garment to create a composite splint.

In some embodiments the garment may include elements (e.g., a plastic underfoot plate, a formable rigid structure (e.g., a SAM splint, or honeycomb) (FIG. 4) for additional structural integrity as needed.

In some embodiments, the filler source is a pressurized dispenser of a curable expanding foam. In some embodiments, the filler component to aid in stabilization, such as, but not limited to a foam (e.g., a fast-curing rigid polyurethane foam) that provides near-instant immobilization, a liquid, gel, air, non-air gas, etc.

### BRIEF DESCRIPTION OF THE DRAWINGS

Aspects, features, benefits and advantages of the embodiments described herein will be apparent with regard to the following description, appended claims, and accompanying drawings where:

FIG. 1 is a sagittal plane cutaway of an embodiment of the system disclosed herein. Note: optional plastic weatherproof shell and a slip-on toe “socklette” are not shown.

FIG. 2 is a schematic of an embodiment disclosed herein.

FIG. 3 is a rendering of an embodiment of the garment of the splint system described herein.

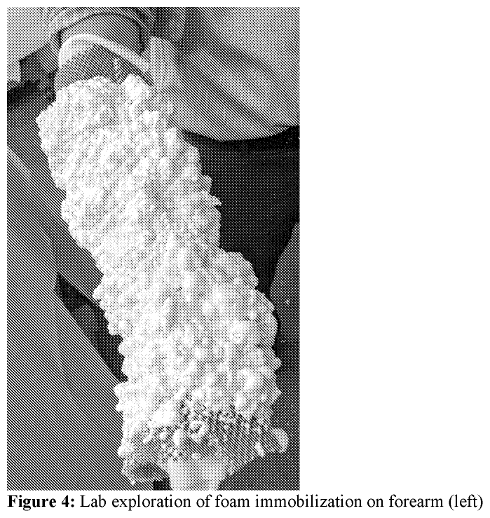

FIG. 4 is a photo depicting application of foam as an independent stabilizing agent without the garment-based splint system described herein wherein the foam has been integrated with additional structural material to create a composite splint.

FIGS. 5-7 illustrate embodiments adapted for various body parts.

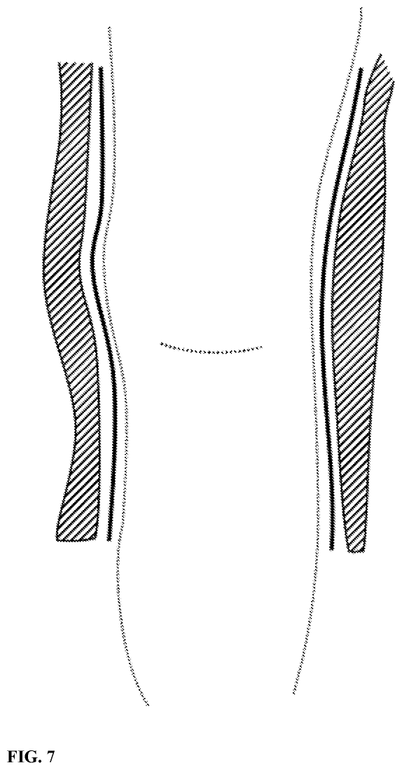

### DETAILED DESCRIPTION

This disclosure is not limited to the particular systems, devices and methods described, as these may vary. The terminology used in the description is for the purpose of describing the particular versions or embodiments only, and is not intended to limit the scope.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also understood that this disclosure is not limited to particular compositions, methods, apparatus, and articles, as these may vary. It is also to be understood that the terminology used herein is for the purpose of describing particular embodiments only, and is not intended to be limiting.

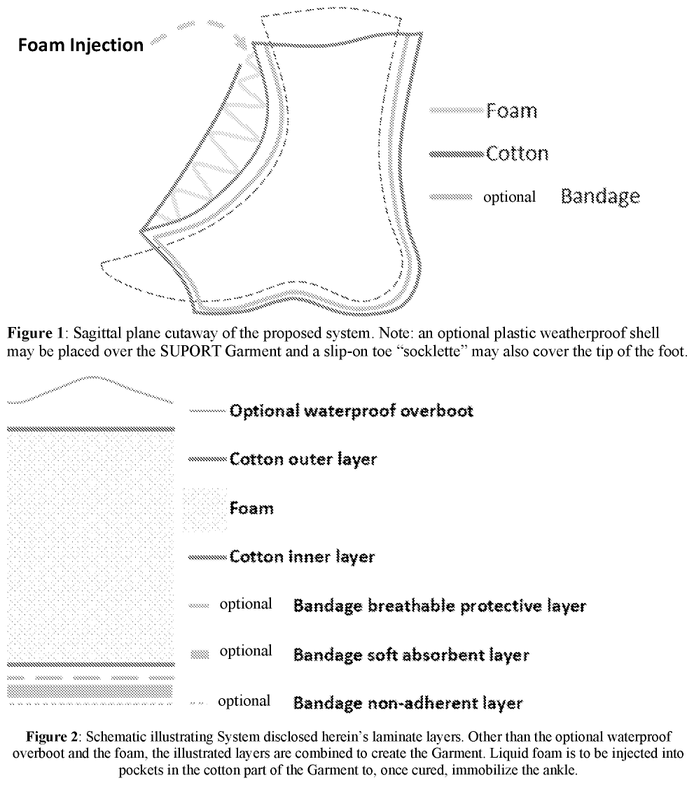

Generally speaking, the system disclosed herein and as depicted in FIG. 1. provides a garment-based splint system, that has several layers, including an inner fabric layer, a filler layer, and an outer fabric layer. Optionally, the system may include a bandage layer in contact with a wound, an outer waterproof outer covering and/or a socklette to cover unprotected areas such as toes. The system is depicted and described herein with reference to a system for addressing an ankle injury, but can be adapted to address any injured joint. In some embodiments, the inner fabric layer and the outer fabric layer form one or more pockets, and one or more channels, wherein filler material may be injected into the one or more pockets via the one or more channels. In some embodiments, the system may be removable via a slit or opening and secured by bands (such as Velcro bands).

Some benefits of the system disclosed herein include but are not limited to one or more of the following: protecting underlying tissue from direct contact with filler (e.g., foam), simplifies filler application by channeling filler to desired fixed volume pockets, locates filler (e.g., foam) to be cured to optimally limit body segment movement with respect to join motion, for example, but not limited to, foot motion with respect to the lower leg, increases cured foam surface strength through integration with garment fibers to create composite layers, provides moisture wicking for tissue health, accommodates underlying tissue swelling and reduction, allows polytrauma wound inspection and management, and more. Further, by controlling the volume of the unfilled pocket, the elasticity of the pocket material, and/or the quantity and material properties (e.g., how much pressure it exerts while expanding and how much it expands) of the filler, the density, hardness, strength, integration with sleeve pockets, or other properties of the resulting structure may be modified to achieve specific results.

It is contemplated that some embodiments herein will not require a barrier layer to protect the skin since some materials which do not negatively impact the skin may be employed.

Use of the sleeve system disclosed herein is easy to apply (does not require a medic or special skills), requires less manipulation of an injury required to apply, allows faster application, provides improved stable immobilization of an injured body segment or joint (e.g. ankle), is transparent to medical imaging technology, enables ease of integration with structural support orthosis elements such as that disclosed in co-pending US Provisional Application entitled Orthosis System filed concurrently herewith and incorporated herein by reference, works with either left or right legs, that is, there is no specific right or left, works for a variety of differently sized individuals, and more. It has been conceived that additional wound treatment layer(s), between the skin and the system disclosed herein, may be used to properly treat polytrauma. The manner in which the foam expands to surround the underlying tissue and material would accommodate any such additional bandage.

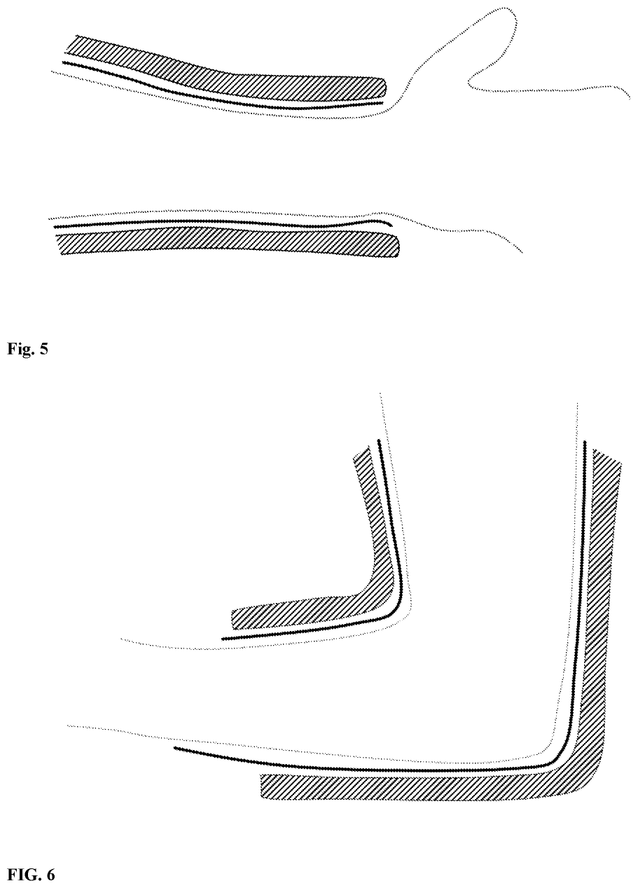

The system disclosed herein uses a combination of a garment plus a filler, such as a foam. Existing systems employ a foam only approach. FIG. 4 shows an expanding foam applied to a forearm without the garment described herein. Undesirable appearance and unevenness of the foam are among the many issues the current system addresses. Further, it is functionally uncontrollable. Meaning, applying foam randomly might lead to a less-effective foam distribution. Exposed foam could be damaged or cause negative effects with other objects/the environment. The foam is sticky and non-solid at first. The garment allows the foam to cure with less possible detrimental interaction with the environment (dirt, equipment, other body parts).

This description will focus on expanding curable foam as the filler, although other fillers are contemplated, such as liquids, gels, air, non-air gasses, etc.

Foam cured in a garment, as disclosed herein, is advantageous over foam alone for several reasons, including but not limited to skin protection, less wasted foam, faster application with less skill, and stronger resulting joint/segment immobilization. Use of garment disclosed herein is different from direct foam application in that it directs and contains foam in an optimized manner predetermined volumes/shapes of cured foam thus result in locations designed to immobilize and protect an injured limb, in some embodiments, once the foam has cured, the garment can be removed and reapplied using securing straps, protects underlying tissue from direct contact with the foam, wicks moisture away from skin, prevents uncured foam ingredients from contacting other garments or equipment, etc.

The garment with foam is also different from other joint immobilization approaches. Powered exoskeletons are bulky, heavy, expensive, and require complex adjustment to fit an injured person's body. Formable splints and braces are not able to fully immobilize an injured limb, especially during subsequent movement, which increases the risk of re-injury. Vacuum splints are very heavy and render the casualty non-ambulatory. Immobilizing walking boots are large and heavy. Simple braces and wraps can effectively treat minor sprains but are not well suited for more severe injuries. Air-casts require additional hardware (a pump) to apply and are not designed for use in austere environments. Plaster and or fiberglass casts require advanced skills to apply, copious pack volume, and significantly more time to apply and set than this garment. Pinned fixators require advanced training and specialized tools to apply, at times including imaging equipment, and necessitate puncturing the skin which can lead to additional complications.

The System embodiment disclosed herein differs from other approaches by being: compact and light, applicable in less time, with less skill, using no tools or power source, and with less manipulation of an injured limb, conformable, fitting both left and right lower limbs for a wide range of individuals (5th percentile female to 95th percentile male), better able to prevent undesirable motion of an injured limb, able to integrate with orthotic structural components, a protective barrier layer between soft tissue (skin and/or wounds) and foam and between foam and the environment (other clothing, equipment, and environmental hazards that could otherwise compromise the foam), adjustable to accommodate swelling, removable for wound inspection and/or management, etc. Many of these benefits are seen because the foam expands to encapsulate the underlying tissue such that a one-size-fits-all approach can work. The foam makes the garment fit.

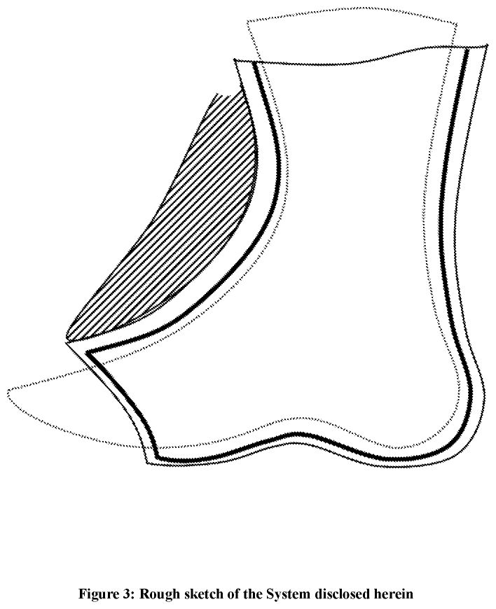

The System embodiment disclosed herein, which is designed to work equally well for both left and right leg injuries, is constructed as a quilted laminate with layers as illustrated, in FIG. 2.

Looking at FIG. 2, each layer will be described below.

The bandage layer is optional and is closest to the skin. It may be any suitable bandage such as a cotton or bamboo-based (or synthetic structured layer) wicking layer. Some embodiments include a non-adherent layer and an absorbent layer. One such bandage is the BLAST brand bandage, which is commonly found in military medical kits.

In one embodiment, BLAST® Bandage's wound pad material, which is typically used as a wrap for polytrauma, may form the base layer. BLAST® Bandage includes three integrated sub-layers: a non-adherent, sterile surface coating; a soft, absorbent layer; and a more durable breathable top layer. BLAST® Bandage is commonly found in med kits and is typically vacuum sealed to reduce pack volume and maintain sterility. Not only do the BLAST® Bandage layers provide a barrier between uncured foam and skin, but they also enhance tissue health due to breathability, potential to wick blood and moisture away from the skin, and physical protection provided to any wounded regions.

The inner fabric layer and the outer fabric layer may be any suitable fabric capable of containing the filler. This allows containment while promoting curing, which helps locate and shape the filler material, especially expanding foam during the curing process. Integration between foam and material fibers is desirable. Suitable fabrics include but are not limited to cotton, flannel, synthetic fabrics, neoprene, nylon (e.g. rip-stop nylon), spandex, mosquito netting, burlap, etc. Together, the inner and outer fabric layers form a structured garment defining at least one channel and at least one pocket for filler delivery and containment. The inner and outer layers may be the same or different materials. In some embodiments, a cotton wicking layer with a moisture barrier touches the skin followed by a cotton flannel layer and an outer cotton flannel pocket covering. It was also observed that a breathable layer helps with foam curing—thus many of these are porous. At least one channel is connected to at least one pocket to allow filler to flow from a port end of the channel to at least one pocket. Where a plurality of pockets is present, they may be interconnected by one or more additional channels to facilitate flow of filler from one pocket to another. In this manner, a series of channels and pockets may be used to ensure proper filling of each pocket. The channels may be separately defined with straw-like tubes, mylar tubes (so they can fold flat), material tubes, plastic conduit, etc.

The filler layer is housed within the one or more pockets formed by the inner and outer fabric layers. Any suitable filler may be used, such as expanding foam, liquid, gel, air, non-air gas, etc.

Together, the inner and outer fabric layers and the filler contained therein work together to support body segment or joint (e.g. the ankle joint). For example, when foam is injected into and allowed to cure within a strategically placed pocket(s) a rigid garment is formed which stabilizes the injured joint or body segment.

In some embodiments, the system may have one or more additional features to improve functionality. For example, in some embodiments, the garment is attached along its bottom edges to a foot-shaped sole form. In some embodiments, it is open along a portion, such as its posterior edges (along the back of the leg) to ease application or removal. It may also have an open toe, finger, thumb or opening to accommodate other such anatomical features, such that it can be placed onto the lower limb and foot from front to back before any foam has been injected. This placement method requires relatively little manipulation of the injured limb. The open region allows easy inspection for circulation and appropriate sensory function without garment removal after the foam has cured. The garment is held in place with adjustable straps (e.g., Velcro) across the back of the leg to accommodate a variety of person sizes. The foam injection process is simplified via easily identifiable openings in the garment (FIG. 1). Channels between the fabric layers direct uncured foam into the structure of the garment such that the foam will expand to fill the supporting pockets substantially completely.

After the foam has cured, in some embodiments, the garment is removable and adjustable for wound inspection and management, even allowing for sub-garment bandages to be replaced as necessary, and to accommodate swelling by way of closure straps. The outer pocket layer protects otherwise softer cured foam from the environment while the inner layer provides an additional barrier between uncured foam and the skin. Cotton flannel is well-suited for this use, since is resistant to foam penetration during injection, expansion, and curing while also permitting air and water vapor to reach uncured foam which facilitates the curing process for a more homogeneous result. Finally, cotton flannel surface fibers integrate well with uncured foam which leads to fibrous reinforced cotton-foam interface layers—effectively creating a composite which adds strength to both the inner and outer surfaces of the resulting structured pockets. Other fabrics or supplemental materials may be employed to achieve similar fibrous reinforcement.

The foot-shaped sole form underfoot portion of the system disclosed herein may include cotton upper and lower layers covering a semi-rigid plastic platform to maintain structural integrity. For additional comfort, orthotic arch supports may be included which can be inserted on the medial-upper surface of the sole prior to foam injection.

An independent, soft toe covering “socklette” (for wicking and warmth) that is easily removed for inspection may be included.

An optional plastic boot cover with an elastic cuff is also included. This part of the garment provides water and weather resistance for the other layers disclosed herein.

Additional elements may be used as part of the system for improved usability, comfort, or other effect.

A rigid toe capsule for protection may be used in conjunction with the system. In some embodiments, this toe covering will be the very outermost layer (even over the waterproof bootie) for physical protection of the toes.

The system may also include attachment points or mechanisms for connecting to an orthosis device that may be used in conjunction with the system disclosed herein.

The one or more channels may take the form of integrated foam distribution tubes to allow fewer foam entry ports. Filler (e.g., foam) can be distributed evenly via a manifold, to distribute foam to pockets evenly. This is imagined to use multiple foam delivery openings inside the pockets. These tubes could remain in place inside the garment, could be something like mylar tubes (no need to be rigid straws), and could have a quick-connect one way valve to prevent foam back-flow. If done correctly, these should distribute foam to the furthest pocket areas first.

The system described above was described in relation to its use in addressing an ankle injury. The system, however, can be easily adapted for a wide variety of joints and areas, including, but not limited to, injury to the lower limb, ankle sprains and fractures, calcaneus fractures, fibula fractures, pilon fractures, tibia fractures, knee sprains, etc. The system can be easily adapted for a wide variety of joints and areas, including, but not limited to, injury to the upper limb, such as humerus fracture, radius fracture, ulna fracture, wrist sprain, etc. The system can be easily adapted for a wide variety of joints and areas, including, but not limited to, injury to other areas of the body, such as the neck and spine, and could even be used on wounds, without a sprain, fracture or other bone or ligament trauma.

In such embodiments, the inner and outer fabric liner could take any shape, such as a tube, capable of substantially enveloping the area requiring treatment or stabilization. The one or more pockets and one or more channels and other features would be sized and placed appropriately for the particular needs of the area to be treated.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of or “consist of the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As used herein, the term “about” means plus or minus 10% of the numerical value of the number with which it is being used. Therefore, about 50% means in the range of 45%55%.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A garment-based splint system comprising:
a garment comprising
an inner fabric layer having a skin-facing surface layer and an interior surface layer, and
an outer fabric layer,
wherein the inner fabric layer and the outer fabric layer define one or more pockets and one or more channels connecting a filler source to the one or more pockets.

2. The garment-based splint system of claim 1, further comprising one way quick disconnect at an end of at least one of the one or more channels adapted for connection with a filler source to allow filler to flow through the one or more channel into the one or more pocket, and to allow removal of the filler source.

3. The garment-based splint system of claim 1, wherein the garment is shaped and configured to stabilize body part, such as the neck, back, an upper limb or a lower limb.

4. A kit for a garment-based splint system the kit comprising:
a garment comprising
an inner fabric layer having a skin-facing surface and an interior surface, and
an outer fabric layer,
wherein the inner fabric layer and the outer fabric layer define one or more pockets and one or more channels connecting a filler source to the one or more pockets; and
a filler source.

5. The kit according to claim 4, wherein the filler source is a pressurized dispenser of a curable expanding foam.
